(19) United States
(12) Patent Application Publication
(10) Pub. No.: US 2025/0131561 A1
(43) Pub. Date: Apr. 24, 2025

(54) Method of Generating Standardized Cerebrovascular Structure Information and Analysis Device

(71) Applicant: SAMSUNG LIFE PUBLIC WELFARE FOUNDATION, Seoul (KR)

(72) Inventors: Woo Keun Seo, Seoul (KR); Jong Un Choi, Seoul (KR); Ha Na Song, Seoul (KR); In Young Baek, Seoul (KR); Ji Eun Lee, Seoul (KR)

(21) Appl. No.: 18/919,555

(22) Filed: Oct. 18, 2024

(30) Foreign Application Priority Data
Oct. 20, 2023 (KR) 10-2023-0141436

(51) Int. Cl. G06T 7/00

(52) U.S. Cl. CPC G06T 7/0012; G06T 2207/10088; G06T 2207/20081; G06T 2207/30016; G06T 2207/30101

(57) ABSTRACT

The method of generating standardized cerebrovascular structure information includes extracting a plurality of vascular unit structures from a cerebrovascular image of a subject, extracting feature values of each of the plurality of vascular unit structures, classifying the plurality of vascular unit structures into chunks by the feature values of each of the plurality of vascular unit structures, classifying multiple vessel branches composed of vascular unit structures belonging to the same chunk by feature values of each of the vascular unit structures, and dividing at least one of the multiple vessel branches into a predetermined number of segments and setting indices for all the segments or segments at certain intervals among the segments.

### CROSS-REFERENCE TO RELATED APPLICATION

This application claims priority to Korean Patent Application No. 10-2023-0141436, filed Oct. 20, 2023, the disclosure of which is hereby incorporated by reference in its entirety.

### BACKGROUND

### 1. Field of the Invention

The following description relates to a technique for normalizing and analyzing vessel branches in a cerebrovascular image.

### 2. Description of Related Art

It is necessary to identify intracranial vascular structures and their variations to diagnose and treat cerebrovascular diseases such as stroke. Magnetic resonance angiography (MRA) is a widely used tool for evaluating cerebral artery diseases.

### SUMMARY

In one general aspect, there is provided a method of generating standardized cerebrovascular structure information, the method including receiving, by an analysis device, a cerebrovascular image of a subject, extracting, by the analysis device, a plurality of vascular unit structures from the cerebrovascular image, extracting, by the analysis device, feature values of each of the plurality of vascular unit structures, classifying, by the analysis device, the plurality of vascular unit structures into chunks by inputting the feature values of each of the plurality of vascular unit structures into a pretrained first learning model, classifying, by the analysis device, multiple vessel branches composed of vascular unit structures belonging to the same chunk by inputting feature values of each of the vascular unit structures belonging to the same chunk into a pretrained second learning model, and dividing, by the analysis device, at least one of the multiple vessel branches into a predetermined number of segments and setting indices for all the segments or segments at certain intervals among the segments.

In another aspect, there is provided a method of generating standardized cerebrovascular structure information, the method including receiving, by an analysis device, cerebrovascular images of subjects belonging to a population, setting, by the analysis device, indices for at least one vessel branch of each of the subjects using the cerebrovascular images of the subjects, and generating, by the analysis device, cerebrovascular structure information of the population by averaging positions of identical indices in the at least one vessel branch of each of the subjects.

In yet another aspect, there is provided an analysis device for evaluating a subject using standardized cerebrovascular structure information, the analysis device including an input device configured to receive a cerebrovascular image of a subject, a storage device configured to store a first learning model which classifies vascular unit structures into chunks, a second learning model which classifies cerebrovascular branches having vascular unit structures belonging to the same chunk, and standardized cerebrovascular structure information of a population, and an arithmetic device configured to extract a plurality of vascular unit structures from the cerebrovascular image on the basis of geometric features of a three-dimensional (3D) model, classify the plurality of vascular unit structures into chunks by inputting feature values of each of the plurality of vascular unit structures into the first learning model, classify multiple vessel branches composed of the vascular unit structures belonging to the same chunk by inputting feature values of each of the vascular unit structures belonging to the same chunk to the second learning model, assign indices for dividing vascular units belonging to at least one of the multiple vessel branches at identical intervals, and compare a position of an index for the at least one vessel branch of the subject with an index position of the standardized cerebrovascular structure information.

### BRIEF DESCRIPTION OF THE DRAWINGS

The above and other objects, features and advantages of the technology described below will become more apparent to those of ordinary skill in the art by describing exemplary embodiments thereof in detail with reference to the accompanying drawings, in which:

FIG. 1 shows an example of a system for extracting features of a subject's cerebral artery structure;

FIG. 2 shows an example of a process of classifying cerebral artery branches;

FIG. 3 is a diagram illustrating a process of training and validating a model used for classifying cerebral artery branches;

FIG. 4 shows a result of predicting chunks of a healthy control group;

FIG. 5 shows a result of predicting chunks of an intracranial atherosclerosis (ICAS) group;

FIG. 6 shows a result of predicting chunks of a stroke group;

FIG. 7 is a set of receiver operating characteristic (ROC) curves showing performance for a stroke group;

FIG. 8 is a set of ROC curves showing performance for an ICAS group;

FIG. 9 shows results of classifying cerebral artery branches of a normal control group;

FIG. 10 shows results of classifying cerebral artery branches of an ICAS group;

FIG. 11 shows results of classifying cerebral artery branches of a stroke group;

FIG. 12 shows an example of a process of quantifying a cerebrovascular structure;

FIG. 13 shows an example of indexing of one vessel branch;

FIG. 14 shows an example of a process of standardizing information on cerebral artery branches;

FIG. 15 is a diagram illustrating another example of a process of standardizing information on cerebral artery branches;

FIG. 16 is a flowchart illustrating a process of evaluating a cerebrovascular structure of a subject using a cerebrovascular standard template; and

FIG. 17 shows an example of an analysis device for analyzing cerebral artery branches of a subject.

### DESCRIPTION OF THE INVENTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

The presently described examples will be understood by reference to the drawings, wherein like parts are designated by like numerals throughout. The drawings are not necessarily drawn to scale, and the size and relative sizes of the layers and regions may have been exaggerated for clarity.

It will be understood that, although the terms first, second, A, B, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

As used herein, including in the claims, “or” as used in a list of items (e.g., a list of items prefaced by a phrase such as “at least one of” or “one or more of” or “one of both of”) indicates an inclusive list such that, for example, a list of at least one of A, B, or C means A or B or C or AB or AC or BC or ABC (i.e., A and B and C).

Before starting detailed explanations of figures, components that will be described in the specification are discriminated merely according to functions mainly performed by the components or conventionally carried out according to common knowledge of related technical fields. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

The technology described below is a technique for quantifying a cerebral artery structure of a subject using a magnetic resonance angiography (MRA) image of the subject's cerebral arteries. The technology described below is a technique for characterizing a cerebral artery structure on the basis of cerebral artery branches.

According to the technology described below, a cerebral artery MRA image is analyzed to classify a vascular structure in units of cerebral artery branches. According to the technology described below, features of a subject's cerebral artery structure are extracted on the basis of the subject's cerebral artery branches. Features of a cerebral artery structure are defined as cerebral artery branch information extracted from a specific subject's cerebral artery MRA image. Each phenotype group (a disease group, a normal group, or the like) of brain diseases has unique features of a cerebral artery structure. The rationale for this will be described below.

The researcher analyzed an image using time-of-flight (TOF) MRA. Accordingly, the technology will be described on the basis of TOF MRA. However, the technology described below may also be applied to other types of medical images.

It is assumed below that an analysis device analyzes a TOF MRA image to classify and standardize cerebral artery branches. The analysis device may uniformly quantify the cerebrovascular structure of each person belonging to a population and generalize the quantified cerebrovascular structure to standardize the cerebrovascular structure. The analysis device may be implemented as various devices for certain data processing. For example, the analysis device may be implemented as a personal computer (PC), a server on a network, a smart device, a chipset with an embedded dedicated program, and the like.

Standardized vessel information is general vascular structure information of a population. For example, standardized vessel information may be average vascular structure information of a specific population. Standardized vessel information may be referred to as a “cerebrovascular standard template.” Here, the population may be defined as phenotypes such as a sex, an age range, and a specific disease. For example, the population may be any one group of healthy people, healthy males, healthy females, people with a cerebrovascular disease, males with a cerebrovascular disease, females with a cerebrovascular disease, people with a specific disease, males with a specific disease, and females with a specific disease.

Also, the analysis device may evaluate a specific subject's cerebrovascular structure using the cerebrovascular standard template.

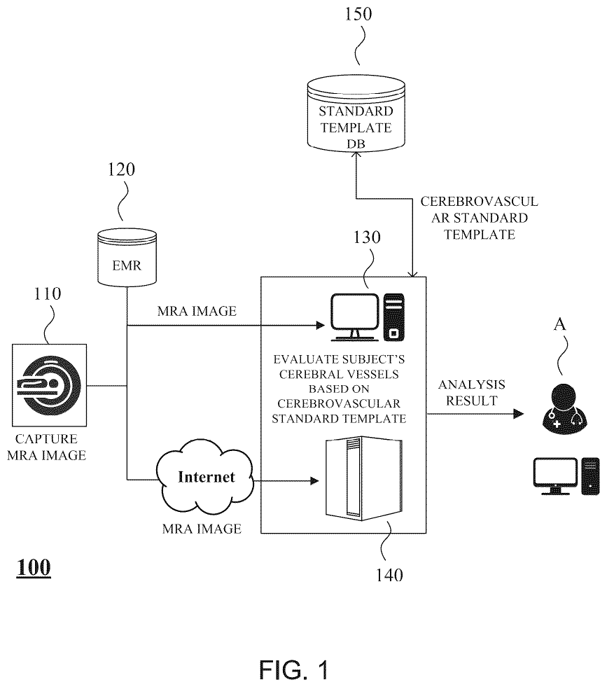

FIG. 1 shows an example of a system 100 for evaluating a cerebral artery structure of a subject. In the example of FIG. 1, an analysis device is a computer terminal 130 and a server 140.

MRA equipment 110 generates an MRA image of a subject. The MRA image or TOF MRA image generated by the MRA equipment 110 may be stored in an electronic medical record (EMR) 120 or a separate database (DB).

The computer terminal 130 may receive the MRA image from the MRA equipment 110 or the EMR 120 via a wired or wireless network. In some cases, the computer terminal 130 may be a device physically connected to the MRA equipment 110. The computer terminal 130 may extract a certain cerebrovascular structure from the MRA image and input features of the extracted cerebrovascular structure to a previously built learning model to derive a cerebral artery branch classification result. The computer terminal 130 may quantify classified cerebral artery branches. Here, the quantification corresponds to a process of extracting features of each of certain sections to identify the structure of each cerebral artery branch. The structure of quantified cerebral artery branches may be defined on the basis of positions of consecutive indices. A detailed quantification process will be described below. The computer terminal 130 may compare the quantified cerebrovascular structure of the subject with a cerebrovascular standard template of a population to which the subject belongs to evaluate the subject's cerebrovascular structure. A standard template DB 150 stores cerebrovascular standard templates of one or more populations. A cerebrovascular standard template generation process will be described below. User A may view an analysis result on the computer terminal 130. The analysis result may include whether the subject's cerebrovascular structure is normal or abnormal.

The server 140 may receive the MRA image from the MRA equipment 110 or the EMR 120. The server 140 may extract a certain cerebrovascular structure from the MRA image and input features of the extracted cerebrovascular structure to a previously built learning model to derive a cerebral artery branch classification result. The server 140 may quantify classified cerebral artery branches. The server 140 may compare the quantified cerebrovascular structure of the subject with a cerebrovascular standard template of the population to which the subject belongs to evaluate the subject's cerebrovascular structure. The server 140 may receive the cerebrovascular standard template of the population from the standard template DB 150. The server 140 may transmit the analysis result to a terminal of user A. User A may view the analysis result on the user terminal.

The computer terminal 130 and/or the server 140 may store the analysis result in the EMR 120.

First, a process of classifying a subject's cerebrovascular structure will be described below. A process in which the researcher builds and validates a model for classifying cerebral artery branches and the validation result will be described.

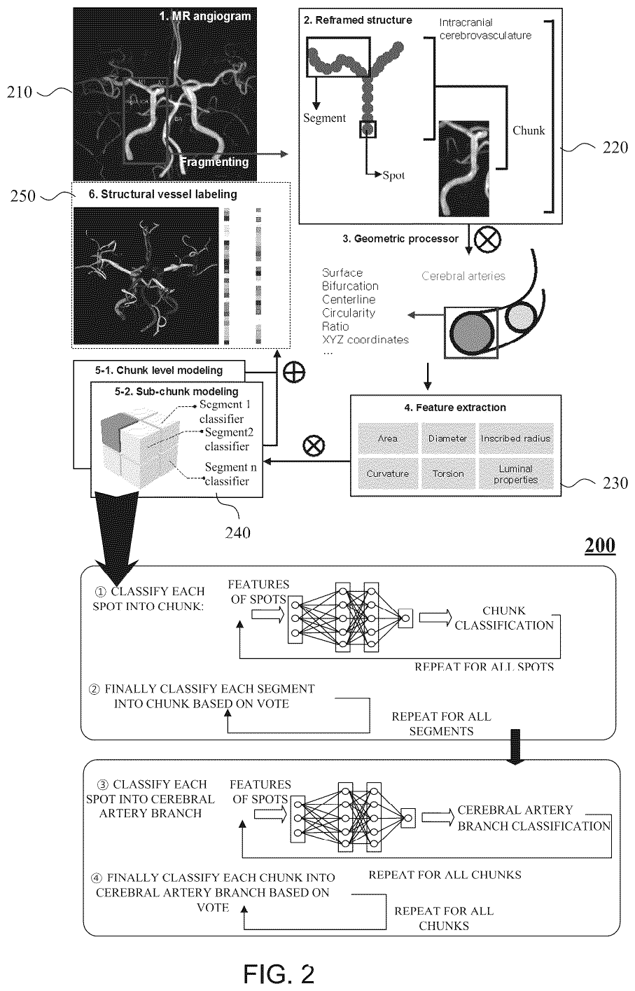

FIG. 2 shows an example of a process 200 of classifying cerebral artery branches. With regard to FIG. 2, a process in which the researcher extracts a vascular structure from a TOF MRA image and classifies vessel branches on the basis of features of the vascular structure will be described. In addition, with regard to FIG. 2, an image processing process and a model building process performed by the researcher will also be described.

The analysis device receives a TOF MRA image (210). The analysis device extracts a structure to extract image features from the TOF MRA image (220). The analysis device may detect desired vascular structures using geometric processing that reconstructs a three-dimensional (3D) model.

The vascular structures include spots, segments, chunks, and branches. The analysis device may extract the vascular structure from the input image using a certain image processing program and extract features of the structure.

First, the vasculature system used by the researcher will be described. The researcher configured vascular unit structures at four hierarchical levels, which is different from a method used in the clinical neurology field according to the related art.

A spot is a basic unit of a 3D cerebral artery tree cubic cell with a regular interval (size) from the arterial centerline. The researcher defined a spot as a cubic cell with an interval of 0.2801 mm from the arterial centerline.

A segment is composed of multiple spots which are divided on the basis of bifurcations in the vascular structure. In other words, a segment corresponds to a set of consecutive spots between bifurcations of a blood vessel.

A vessel branch is composed of a plurality of segments. A vessel branch may be identified as one of specific types on the basis of segments according to the geometry of a vascular bifurcation. The researcher classified vessel branches into 62 types of branches according to the geometry of vascular bifurcations. In other words, vessel branches may be uniformly classified according to the vascular unit structures proposed by the researcher. Meanwhile, a vessel branch may be identified using a nomenclature traditionally used in the clinical field.

A vessel branch may be segmented into (i) symmetry, (ii) anterior or posterior, (iii) basal or pial, and (iv) middle cerebral arteries (MCA), anterior cerebral arteries (ACA), or posterior cerebral arteries (PCA) according to clinical criteria. Based on such segmentation, the researcher defined a chunk as a higher-level structure than a branch. A chunk may be classified into a type using at least one criterion among groups including (i) symmetry, (ii) anterior or posterior, (iii) basal or pial, and (iv) MCA, ACA, and PCA. In other words, a chunk is clinically defined as vessel branches of a higher-level mass. The reason for defining a chunk is because it is difficult to classify branch units at once due to a detailed structure of a vessel branch.

The researcher defined 20 types of vascular chunks as shown in Table 1 below. Each chunk may be further subdivided into one or more branches.

TABLE 1

| | | Chunk | | Branch |
|---|---|---|---|---|
| Number | Chunk (abbreviation) | code | Branch (abbreviation) | code |
| 1 | Anterior communicating | A0 | anterior communicating artery | A0.01 |
| | artery | | (ACoA) | |
| | (ACOA) | | | |
| 2 | Right internal carotid artery | A1 | Right internal carotid artery | A1.01 |
| | (RtICA) | | (Rt ICA) | |
| | | | Right ophthalmic artery | A1.02 |
| | | | (Rt Ophthalmic) | |
| | | | Right anterior choroidal artery | A1.03 |
| | | | (Rt AChA) | |
| 3 | Left internal carotid artery | A2 | Left internal carotid artery | A2.01 |
| | (LtICA) | | (Lt ICA) | |
| | | | Left ophthalmic artery | A2.02 |
| | | | (Lt Ophthalmic) | |
| | | | Left anterior choroidal artery | A2.03 |
| | | | (Lt AChA) | |
| 4 | Right anterior cerebral basal | A3 | Right MCA M1 | A3.01 |
| | arteries - middle cerebral | | (Rt M1) | |
| | artery | | Right M2 superior | A3.02 |
| | (RtBasalMCA) | | (Rt MCA Superior) | |
| | | | Right M2 inferior | A3.03 |
| | | | (Rt MCA Inferior) | |
| 5 | Left anterior cerebral basal | A4 | Left anterior basal MCA | A4.01 |
| | arteries - middle cerebral | | (Lt M1) | |
| | artery | | Left_MCA_Superior | A4.02 |
| | (LtBasalMCA) | | (Lt MCA Superior) | |
| | | | Left_MCA_Inferior | A4.03 |
| | | | (Lt MCA Inferior) | |
| 6 | Right anterior cerebral basal | A5 | Right ACA A1 | A5.01 |
| | arteries - anterior cerebral | | (Rt A1) | |
| | artery | | Right ACA A2 | A5.02 |
| | (RtBasalACA) | | (Rt A2) | |
| | | | Right ACA A1 + A2 | A5.03 |
| | | | (Rt A1 + A2) | |
| 7 | Left anterior cerebral basal | A6 | Left ACA A1 | A6.01 |
| | arteries - anterior cerebral | | (Lt A1) | |
| | artery | | Left ACA A2 | A6.02 |
| | (LtBasalACA) | | (Lt A2) | |
| | | | Left ACA A1 + A2 | A6.03 |
| | | | (Lt A1 + A2) | |
| 8 | Right anterior cerebral pial | A7 | Right orbitofrontal artery | A7.01 |
| | arteries - middle cerebral | | (Rt MCA lat OFA) | |
| | artery | | Right MCA PreRolandic artery | A7.02 |
| | (RtPialMCA) | | (Rt MCA PreRolandic) | |
| | | | Right MCA Rolandic artery | A7.03 |
| | | | (Rt MCA Rolandic) | |
| | | | Right MCA Anterior Parietal | A7.04 |
| | | | artery | |
| | | | (Rt MCA AntPerietal) | |
| | | | Right MCA Posterior Parietal | A7.05 |
| | | | artery | |
| | | | (Rt MCA PostParietal) | |
| | | | Right MCA Angular artery | A7.06 |
| | | | (Rt MCA Angular) | |
| | | | Right MCA Posterior Temporal | A7.07 |
| | | | artery | |
| | | | (Rt MCA PostTemporal) | |
| | | | Right MCA MidTemporal | A7.08 |
| | | | artery | |
| | | | (Rt MCA MidTemporal) | |
| | | | Right MCA Anterior temporal | A7.09 |
| | | | artery | |
| | | | (Rt MCA AntTemporal) | |
| 9 | Left anterior cerebral pial | A8 | Left MCA orbitofrontal artery | A8.01 |
| | arteries - middle cerebral | | (Lt MCA lat OFA) | |
| | artery | | Left MCA PreRolandic artery | A8.02 |
| | (LtPialMCA) | | (Lt MCA PreRolandic) | |
| | | | Left MCA Rolandic artery | A8.03 |
| | | | (Lt MCA Rolandic) | |
| | | | Left MCA Anterior Parietal | A8.04 |
| | | | artery | |
| | | | (Lt MCA AntParietal) | |
| | | | Left MCA Posterior Parietal | A8.05 |
| | | | artery | |
| | | | (Lt MCA PostParietal) | |
| | | | Left MCA Angular artery | A8.06 |
| | | | (Lt MCA Angular) | |
| | | | Left MCA Posterior Temporal | A8.07 |
| | | | artery | |
| | | | (Lt MCA PostTemporal) | |
| | | | Left MCA MidTemporal artery | A8.08 |
| | | | (Lt MCA MidTemporal) | |
| | | | Left MCA Antior temporal | A8.09 |
| | | | artery | |
| | | | (Lt MCA AntTemporal) | |
| 10 | Right anterior cerebral pial | A9 | Right ACA orbitofrontal artery | A9.01 |
| | arteries - anterior cerebral | | (Rt ACA med OFA) | |
| | artery | | Right ACA Frontopolar artery | A9.02 |
| | (RtPialACA) | | (Rt A2 Frontopolar) | |
| | | | Right ACA Callosomarginal | A9.03 |
| | | | artery | |
| | | | (Rt ACA Callosomarginal) | |
| | | | Right ACA Pericallosal artery | A9.04 |
| | | | (Rt ACA Pericallosal) | |
| 11 | Left anterior cerebral pial | A10 | Left ACA orbitofrontal artery | A10.01 |
| | arteries - anterior cerebral | | (Lt ACA med OFA) | |
| | artery | | Left ACA Frontopolar artery | A10.02 |
| | (LtPialACA) | | (Lt A2 Frontopolar) | |
| | | | Left ACA Callosomarginal | A10.03 |
| | | | artery | |
| | | | (Lt ACA Callosomarginal) | |
| | | | Left ACA Pericallosal artery | A10.04 |
| | | | (Lt ACA Pericallosal) | |
| 12 | Right posterior - vertebral | P1 | Right vertebral artery | P1.01 |
| | artery | | (Rt VA) | |
| | (RtVA) | | | |
| 13 | Left posterior - vertebral | P2 | Left vertebral artery | P2.01 |
| | artery | | (Lt VA) | |
| | (LtVA) | | | |
| 14 | Right posterior basal - | P3 | Right PCA P1 | P3.01 |
| | posterior cerebral artery | | (Rt P1) | |
| | (RtBasalPCA) | | Right PCA P2 | P3.02 |
| | | | (Rt P2) | |
| | | | Right PCA P1, P2 | P3.03 |
| | | | (Rt P1 + P2) | |
| | | | Right PCA P3, P4 | P3.04 |
| | | | (Rt P3, P4) | |
| 15 | Left posterior basal - | P4 | Left PCA P1 | P4.01 |
| | posterior cerebral artery | | (Lt P1) | |
| | (LtBasalPCA) | | Left PCA P2 | P4.02 |
| | | | (Lt P2) | |
| | | | Left PCA P1 + P2 | P4.03 |
| | | | (Lt P1 + P2) | |
| 16 | Right posterior pial - | P5 | Right posterior communicating | P5.01 |
| | posterior cerebral artery | | artery | |
| | (RtPialPCA) | | (Rt PCoA) | |
| | | | Right Hippocampal artery | P5.02 |
| | | | (Rt Hippocampal artery) | |
| | | | Right PCA Anterior Temporal | P5.03 |
| | | | artery | |
| | | | (Rt PCA AnteriorTemporal) | |
| | | | Right PCA Posterior Temporal | P5.04 |
| | | | artery | |
| | | | (Rt PCA PosteriorTemporal) | |
| | | | Right parieto-occipital artery | P5.05 |
| | | | (Rt parieto-occipital) | |
| | | | Right calcarine artery | P5.06 |
| | | | (Rt Calcarine) | |
| 17 | Left posterior pial - | P6 | Left posterior communicating | P6.01 |
| | posterior cerebral artery | | artery | |
| | (LtPialPCA) | | (Lt PCoA) | |
| | | | Left_Hippocampal artery | P6.02 |
| | | | (Lt Hippocampal artery) | |
| | | | Left_PCA_Anterior | P6.03 |
| | | | Rightemporal | |
| | | | (Lt PCA AnteriorTemporal) | |
| | | | Left_PCA_Posterior | P6.04 |
| | | | Rightemporal | |
| | | | (Lt PCA PosteriorTemporal) | |
| | | | Left parieto-occipital artery | P6.05 |
| | | | (Lt parieto-occipital) | |
| | | | Left calcarine artery | P6.06 |
| | | | (Lt Calcarine) | |
| 18 | Right posterior - superior | P7 | Right posterior inferior | P7.01 |
| | cerebral artery, anterior | | cerebellar artery | |
| | inferior cerebral artery, | | (Rt PICA) | |
| | posterior inferiorcerebellar | | Right anterior inferior | P7.02 |
| | artery | | cerebellar artery | |
| | (RtCbll) | | (Rt AICA) | |
| | | | Right internal auditory artery | P7.03 |
| | | | (Rt IAA) | |
| | | | Right superior cerebellar artery | P7.04 |
| | | | (Rt SCA) | |
| 19 | Left posterior - superior | P8 | Left posterior inferior | P8.01 |
| | cerebral artery, anterior | | cerebellar artery | |
| | inferior cerebral artery, | | (Lt PICA) | |
| | posterior inferiorcerebellar | | Left anterior inferior cerebellar | P8.02 |
| | artery | | artery | |
| | (LtCbll) | | (Lt AICA) | |
| | | | Left internal auditory artery | P8.03 |
| | | | (Lt IAA) | |
| | | | Left superior cerebellar artery | P8.04 |
| | | | (Lt SCA) | |
| 20 | Basilar artery (BA) | P0 | Basilar artery | P0.01 |
| | | | (BA) | |

The researcher acquired images of the intracranial arteries on a 3.0 T Philips Achieva magnetic resonance imaging (MRI) scanner (Philips Medical Systems). The researcher used a whole-brain 3D MRA image with a TOF protocol collected from each participant. The parameters for an isotropic 0.284×0.284 mm³ size voxel are as follows: echo time of 4.59 ms, repetition time of 22 ms, flip angle of 23°, receiver bandwidth (RBW) of 130 Hz/pixel, generalized autocalibrating partially parallel acquisitions (GRAPPA) factor of 3, and 32 reference lines.

The data format used by the researcher was Digital Imaging and Communications in Medicine (DICOM)® in a TOF format. The researcher anonymized the raw data using DICOM Anonymizer Pro, and performed region growing using a vascular analysis program that generates divided cerebral angiography masks.

A process of extracting a vascular surface from a cerebrovascular MRA image will be described. The researcher performed an isosurface dissection to create a vascular surface model using the Vascular Modeling Toolkit (VMTK) library. The researcher removed artifacts using bicubic interpolation and resampled a rough image on a regular flat grid. This enabled a z-axis voxel to be fitted to an isovoxel image scale. A continuous 3D space may be divided into multiple cells based on each vertex of the isosurfaces. Through this process, the researcher may extract the main arterial centerline from the perimeter surface of each cell in the cerebrovascular MRA image.

The analysis device may divide the vascular surface into cells of a certain size in the cerebrovascular MRA image, and extract the start point and skeleton of the centerline of the brain artery on the basis of the vascular surface.

The analysis device may perform vascular skeleton refinement to make the end point of the centerline more distinct. The analysis device may (i) skeletonize the cerebrovascular region and surface, (ii) prune branches under a predetermined threshold, (iii) generate a linked list of tree structures on the basis of the refined skeletal structure, and (iv) determine the end point by specifying a leaf node from the linked list. The analysis device may extract the centerline of the blood vessel by tracing the cell boundary connecting the determined start point and end point.

Subsequently, the analysis device characterizes the vascular feature vectors for groups separated on the basis of the bifurcations of the centerline (230). Vascular features include a cerebrovascular cross-sectional area, a maximally inscribed sphere radius, a minimum diameter, a maximum diameter, a maximum-minimum radius ratio, a surface circumference, torsion, curvature, and luminal circularity.

The researcher performed automated segmentation at the level of general nomenclature of the cerebrovascular system in the brain MRA image, and built a model to perform classification (labeling) for cerebral artery branches. The analysis device classifies the vascular structure at a chunk level using a previously built first learning model, and classifies each chunk into specific branches (cerebral artery branches) using a second learning model (240).

The researcher used a multi-layer perceptron (MLP) as a supervised learning model.

It is self-evident that other models may be used as a learning model for classifying cerebral artery branches. Here, the machine learning model is any of a decision tree, a random forest (RF) model, a K-nearest neighbor (KNN) model, a naive Bayes model, a support vector machine (SVM), an artificial neural network (ANN), and the like. Meanwhile, an ANN is a statistical learning algorithm that imitates a biological neural network. Various neural network models are being studied. A deep neural network (DNN) may model complex non-linear relationships like a general ANN. Various types of DNN models have been studied. For example, the DNN may be a convolutional neural network (CNN), a recurrent neural network (RNN), a restricted Boltzmann machine (RBM), a deep belief network (DBN), a generative adversarial network (GAN), a relation network (RL), etc.

The analysis device first performs classification at a chunk level (Step 1: Chunk level modeling).

The analysis device first inputs the feature vector of each spot into the first learning model in units of spots extracted from the TOF MRA image. Here, the feature vector of the spot may be a value for at least one of the vascular features described above. Further, the feature vector of the spot may further include a brightness value of the spot region.

The researcher used a DNN as the first learning model. The first learning model classifies input spots at a chunk level. This process is repeated for all spots. As a result, the analysis device obtains chunk classification results of all spots (primary classification).

Further, the analysis device may complete the primary chunk classification of all spots, and then perform voting-based classification for accuracy improvement (secondary classification). To this end, the analysis device divides segments on the basis of the bifurcations of blood vessels. This process enables the analysis device to divide segments of the entire blood vessel. The analysis device may perform chunk allocation on the basis of a majority vote in units of segments. The analysis device may check the result of classifying each of spots belonging to the same segment through the first learning model, and determine the most classified result (chunk type) as a final classification result of spots constituting the corresponding segment. This enables the analysis device to classify the chunk to which the corresponding segment (or spots belonging to the corresponding segment) belongs in units of segments. This process may be repeated for all segments.

Here, the analysis device performs sub-classification in units of classified chunks (Step 2: Sub-chunk modeling).

The analysis device inputs each of spots belonging to the same chunk into the second learning model in units of chunks. The second learning model classifies the input spot into a specific cerebral artery branch. This process is repeated for all chunks. As a result, the analysis device acquires cerebral artery branch classification results (primary classification) of all the spots.

Meanwhile, the second learning model may be the same type of model (DNN) as the first learning model. Also, the second learning model may be a different type of machine learning model from the first learning model. Further, the second learning model may be an ensemble model.

Further, the analysis device may complete the cerebral artery branch classification results (primary classification) for all the spots and then perform voting-based classification (secondary classification) for accuracy improvement. The analysis device may perform chunk allocation on the basis of a majority vote in units of chunks. The analysis device may check the result of classifying each of spots belonging to the same chunk through the second learning model, and determine the most classified result (a type of cerebral artery branch) as a final classification result of the spots constituting the corresponding chunk. This enables the analysis device to classify cerebral artery branches (or spots belonging to the corresponding chunk) into a corresponding chunk in units of chunks. This process may be repeated for all chunks.

Meanwhile, the analysis device may validate or correct a cerebral artery branch classification result (primary classification) for the spots in a different method. For example, the analysis device may validate whether segments (branches belonging to the corresponding segments) of which branches are clearly distinguished as left and right or up and down in the image data are in opposite directions on the basis of their 3D coordinates, and correct the classification information when the segments are opposite to each other.

The arithmetic device computes information by performing classification in units of cerebral artery branches in the brain MRA image of the subject (patient) through the process shown in FIG. 2 (250). The medical staff may diagnose a disease and take treatment measures for the subject on the basis of the final classification result.

Table 2 below shows examples of cerebrovascular features used in chunk classification and cerebral artery branch classification.

TABLE 2

| Feature | Description |
|---|---|
| X, Y, Z | Coordinates of vascular centerline |
| Area | Vascular cross-sectional area at specific point |
| Max inscribed | Maximally inscribed sphere radius |
| sphere radius | |
| Min-diameter | Minimum diameter of cross-section at specific point |
| Max-diameter | Maximum diameters of cross-section at specific point |
| Hydraulic | Hydraulic diameter of blood vessel |
| luminal diameter | |
| Perimeter | Circumference of cross-section at specific point |
| Min-max | Maximum-minimum radius ratio |
| radius ratio | |
| Luminal | Luminal roundness |
| circularity | |
| Curvature | Curvature of centerline at specific point |
| Torsion | Torsion of centerline at specific point |

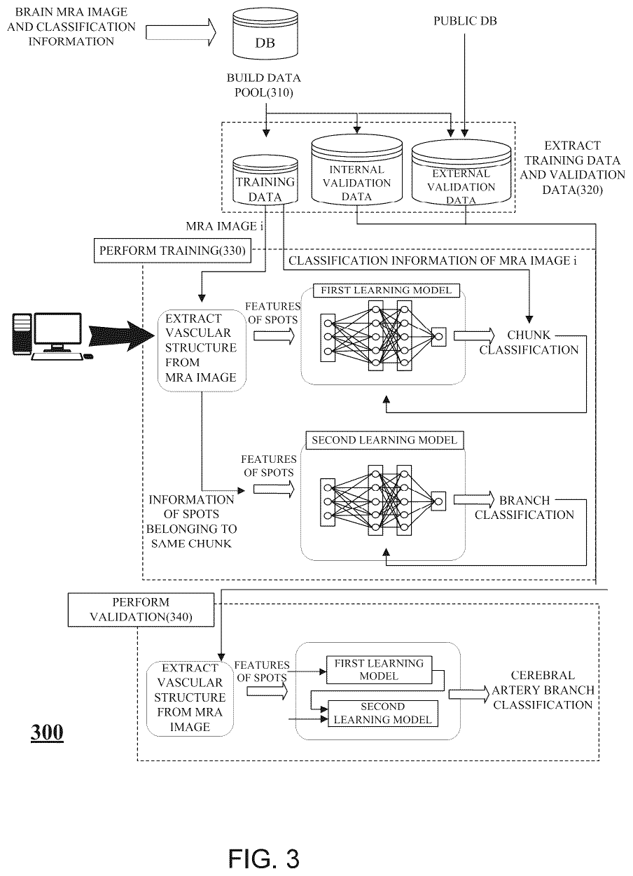

FIG. 3 is a diagram illustrating a process 300 of training and validating a model used for classifying cerebral artery branches.

The researcher studied stroke patients clinically confirmed at Samsung Medical Center after obtaining consent from them. The control cohort is defined as 1,181 people with no disease from whose images features are extractable among those having undergone MRA imaging from Jan. 13, 2014, to Dec. 30, 2016, at the Samsung Hospital Health Examination Center.

Further, the researcher studied a healthy control group, stroke patients with intracranial atherosclerosis (ICAS) (stroke with ICAS, ICAS group), and normal stroke patients (stroke group). The researcher studied 157 participants between the ages of 20 and 94. The 157 participants were divided into the control group with 42 people, the ICAS group with 46 people, and the stroke group with 69 people. The researcher used 70% of the collected cohort data as training data and 30% as validation data.

The researcher analyzed TOF MRA images of the participants to identify the structure of the cerebral artery vessels in advance (image discrimination by experts). In other words, the researcher classified cerebral artery branches in the training data and validation data in advance. The classification information of the cerebral artery branches is simply referred to as classification information hereinafter.

The researcher builds a data pool for training and validation (310). The data pool contains brain MRA images of the participants.

The researcher extracts training data and validation data (320). The researcher prepares the training data and the internal validation data on the basis of the MRA images acquired through the affiliated institution. The training data and the validation data each include a brain MRA image of a specific subject and classification information for the image. In addition, the researcher may perform external validation using images prepared by the corresponding institution. It is self-evident that external validation data may be prepared by collecting MRA images and classification information in an external public DB.

The researcher trains the aforementioned learning model using the training data (330). The learning models are trained on the analysis device or a separate computing device. Hereinafter, a device for building the learning models is referred to as a training device. The training device trains the first learning model and the second learning model using the training data. The training device extracts the aforementioned vascular unit structures from the MRA image. The process of extracting the vascular unit structures is the same as described above. The vascular unit structures include spots, segments, and the like as described above. The training device inputs features in units of spots in the extracted vascular unit structures into the first learning model to classify corresponding spots into chunks. In this process, the training device inputs features of the spots into the first learning model, and then compares a probability value of chunk classification output by the first learning model with classification information of the corresponding spot to update parameters of the first learning model. FIG. 3 illustrates a training process performed using MRA image i, and the training process is repeatedly performed using various training data. By repeating this process, the first learning model is trained to output the chunk classifications of the corresponding spots on the basis of features of the input spots.

Meanwhile, even in the training process of the learning models, the training device may perform voting on the basis of the classification results of spots belonging to each segment, to determine a final classification result of spots belonging to the corresponding segment.

The training device inputs features of each of spots belonging to the same chunk into the second learning model in units of chunks, to classify a cerebral artery branch to which the corresponding spot belongs. Here, input spots are spots belonging to the same chunk. The training device inputs features of spots belonging to the same chunk into the second learning model and compares probability values of cerebral artery branch classifications output by the second learning model with classification information of a corresponding spot, to update parameters of the second learning model. By repeating this process, the second learning model is trained to output the cerebral artery branch classification of a corresponding spot on the basis of features of spots belonging to the same chunk.

Meanwhile, even in the training process of the learning models, the training device may perform voting on the basis of the classification results of spots belonging to each chunk, to determine a final classification result of spots belonging to a corresponding chunk. Subsequently, the training device validates the learning models trained using internal validation data and/or external validation data (340). The training device extracts a vascular unit structure from an input MRA image and inputs features of each spot into the first learning model to classify the corresponding spot into a chunk. Thereafter, the training device inputs features of each spot belonging to the same chunk into the second learning model according to the chunk classification results to classify a cerebral artery branch to which the corresponding spots belong. The researcher checked the performance of the classification results using validation data.

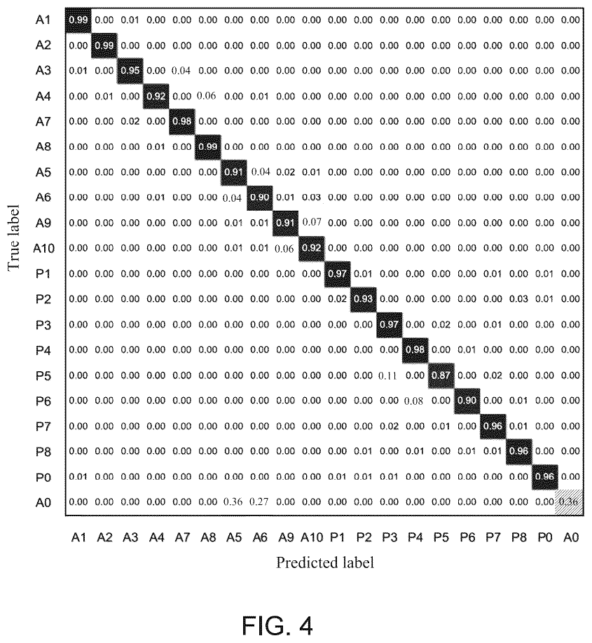

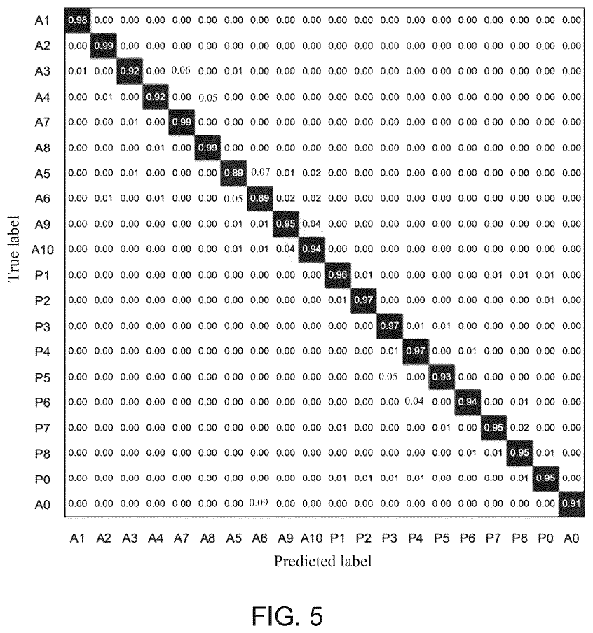

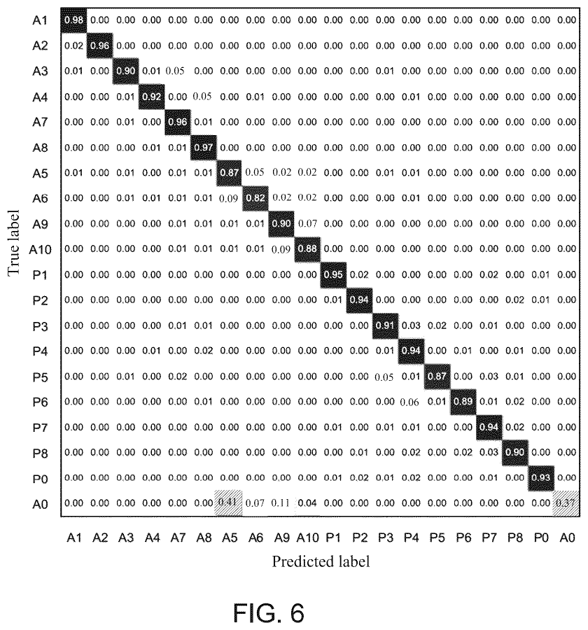

FIGS. 4 to 6 are results of validating the performance of predicting chunks in a cerebrovascular structure. FIG. 4 shows a result of predicting chunks of a healthy control group. FIG. 5 shows a result of predicting chunks of an ICAS group. FIG. 6 shows a result of predicting chunks of a stroke group. In the graphs of FIGS. 4 to 6, the vertical axis indicates a true label, and the horizontal axis indicates a label predicted by the model. A model built by the researcher showed 82% prediction accuracy for only the left anterior basolateral anterior cerebral artery chunks (LtBasalACA, A6) among 20 chunks and showed 87% to 99% prediction accuracy for the rest. In addition, the model built by the researcher showed similar prediction accuracy for each chunk in the healthy control group, the stroke group, and the ICAS group. However, the ACOA (A0) showed some predictive deviation depending on the subject group. This may be due to the limited sample size and high anatomical variability for ACOA. The area under the curve (AUC)-receiver operating characteristic (ROC) curve for chunk classification ranges from 0.99 to 1.00 overall, which is high performance, and the precision-recall curve (PRC) was 0.992.

The researcher also validated the classification accuracy of the model for cerebral artery branches. Table 3 below shows the prediction accuracy of main cerebral artery branches belonging to the 20 chunks as a result of the experiment. Table 3 below shows results of summarizing the entire cohort (control group, ICAS group, and stroke group). When there is only one cerebral artery branch belonging to a chunk, the prediction accuracy for the cerebral artery branch is not indicated.

TABLE 3

| Chunk | | Accuracy | Main cerebral | Accuracy |
|---|---|---|---|---|
| code | Chunk | (%) | artery branch | (%) |
| A1 | Right ICA | 98-99 | Right ICA | 100 |
| | | | Right OA | 94-99 |
| | | | Right ACHA | 85 |
| A2 | Left ICA | 96-99 | Left ICA | 100 |
| | | | Left OA | 91-95 |
| | | | Left ACHA | 91 |
| A3 | Right anterior | 90-95 | Right M1 | 96 |
| | basal MCA | | Right MCAS | 87-91 |
| | | | Right MCAI | 90-96 |
| A4 | Left anterior | 92 | Left M1 | 95-97 |
| | basal MCA | | Left MCAS | 85-92 |
| | | | Left MCAI | 92-94 |
| A7 | Right anterior | 96-99 | Right MCALO | 95 |
| | pial MCA | | right MCAPR | 87-98 |
| | | | right MCAR | 95-97 |
| | | | right MCAAP | 87-95 |
| | | | right MCAPP | 93-95 |
| | | | right MCAA | 94-97 |
| | | | right MCAPT | 90-94 |
| | | | right MCAMT | 93 |
| | | | right MCAAT | 90-94 |
| | | | right MCAPF | 82-85 |
| A8 | Left anterior | 97-99 | Left MCALO | 81 |
| | pial MCA | | left MCAPR | 93-96 |
| | | | left MCAR | 94-96 |
| | | | left MCAAP | 89-94 |
| | | | left MCAPP | 90-94 |
| | | | left MCAA | 94-97 |
| | | | left MCAPT | 92-94 |
| | | | left MCAMT | 91 |
| | | | left MCAAT | 90-92 |
| | | | left MCAPF | 88 |
| A5 | Right anterior | 87-92 | Right A1 | 93-95 |
| | basal ACA | | right A2 | 97-99 |
| | | | right A1A2 | 97 |
| A6 | Left anterior | 82-89 | Left A1 | 94-95 |
| | basal ACA | | left A2 | 96-97 |
| | | | left A1A2 | 76-100 |
| A9 | Right anterior | 88-95 | Right ACAMO | 89 |
| | pial ACA | | Right A2F | 90-96 |
| | | | Right ACAC | 94-97 |
| | | | Right ACAP | 97-98 |
| A10 | Left anterior | 88-94 | Left ACAMO | 88 |
| | pial ACA | | Left A2F | 93-96 |
| | | | Left ACAC | 93-96 |
| | | | Left ACAP | 97-98 |
| P1 | Right posterior VA | 95-97 | Right VA | |
| P2 | Left posterior VA | 94-97 | Left VA | |
| P3 | Right posterior | 91-97 | Right P1 | 86-89 |
| | basal PCA | | Right P2 | 90-92 |
| | | | Right P1P2 | 87-94 |
| | | | Right P3P4 | 98 |
| P4 | Left posterior | 94-97 | Left P1 | 86-90 |
| | basal PCA | | Left P2 | 93-95 |
| | | | Left P1P2 | 94-97 |
| | | | Left P3P4 | 98-99 |
| P5 | Right posterior | 87-93 | Right PPA | 88 |
| | pial PCA | | right HA | NA |
| | | | right PCAAT | 86-93 |
| | | | right PCAPT | 96-100 |
| | | | right PCALP | NA |
| | | | right PCOA | 93-98 |
| P6 | Left posterior | 89-94 | Left PPA | 87 |
| | pial PCA | | left HA | NA |
| | | | left PCAAT | 81-94 |
| | | | left PCAPT | 97-98 |
| | | | left PCALP | 100 |
| | | | left PCOA | 78-95 |
| P7 | Right SCA, | 94-97 | Right PICA | 95-98 |
| | AICA, and | | Right AICA | 94-97 |
| | PICA | | Right IAA | 79 |
| | | | Right SCA | 99 |
| P8 | Left SCA, | 90-97 | Left PICA | 96-98 |
| | AICA, and | | Left AICA | 89-96 |
| | PICA | | Left IAA | NA |
| | | | Left SCA | 98-99 |
| P0 | BA | 93-97 | BA (G190) | |
| A0 | ACOA | 37-91 | ACOA (G200) | |

Abbreviations in Table 3 have the following meanings: internal carotid arteries (ICA), ophthalmic arteries (OA), anterior choroidal arteries (ACHA), vertebral arteries (VA), posterior inferior cerebellar arteries (PICA), anterior inferior cerebellar arteries (AICA), internal auditory arteries (IAA), superior cerebellar arteries (SCA), posterior communicating arteries (PCOA), posterior cerebral arteries (PCA), pre-communicating PCA (P1), post-communicating PCA (P2), the coalescence among P1 and P2 (P1P2), the concoction of quadrigeminal and calcarine PCA (P3P4), direct peduncular perforating arteries (PPA), hippocampal arteries (HA), anterior temporal PCA (PCAAT), posterior temporal PCA (PCAPT), lateral posterior choroidal arteries (PCALP), sphenoidal middle cerebral artery (M1), middle cerebral arteries (MCA), superior division of MCA (MCAS), inferior division of MCA (MCAI), lateral orbitofrontal arteries (MCALO), pre-Rolandic MCA (MCAPR), Rolandic MCA (MCAR), anterior parietal MCA (MCAAP), posterior parietal MCA (MCAPP), angular MCA (MCAA), posterior temporal MCA (MCAPT), middle temporal MCA (MCAMT), anterior temporal MCA (MCAAT), pre-frontal MCA (MCAPF), anterior cerebral arteries (ACA), horizontal pre-communicating ACA (A1), vertical post-communicating pre-callosal ACA (A2), the combination of A1 and A2 (A1A2), medial orbitofrontal ACA (ACAMO), frontopolar vertical post-communicating pre-callosal ACA (A2F), callosomarginal ACA (ACAC), peri-callosal ACA (ACAP), basilar artery (BA), and anterior communicating artery (ACOA). The classification prediction accuracy was examined for each cohort. The prediction accuracy in the control group ranged from 90% to 99% except for right MCAPF, left A1/A2, and left PCAAT. In the ICAS group, the prediction accuracy was 91% to 100% for ICA, 85% to 98% for MCA, 88% to 100% for ACA, 87% to 100% for PCA, and 96% to 99% for SCA-AICA-PICA. In the stroke group, the prediction accuracy was 94% to 100% for ICA, 90% to 96% for MCA, 94% to 98% for ACA, 90% to 99% for PCA, and 96% to 99% for SCA-AICA-PICA. The overall AUC-ROC for cerebral artery branch classification was 0.99, which is high performance, and the PRC was 0.992.

The researcher also performed external validation. The external validation was performed as a validation of the ability to identify stroke patients. The researcher built a learning model using only the data of the control group. In other words, the learning model used for the external validation corresponds to a model that outputs a probability value for stroke patients without classifying cerebral artery branches. The researcher evaluated the ability to distinguish stroke patients in the ICAS group (46 people) and the stroke group (69 people).

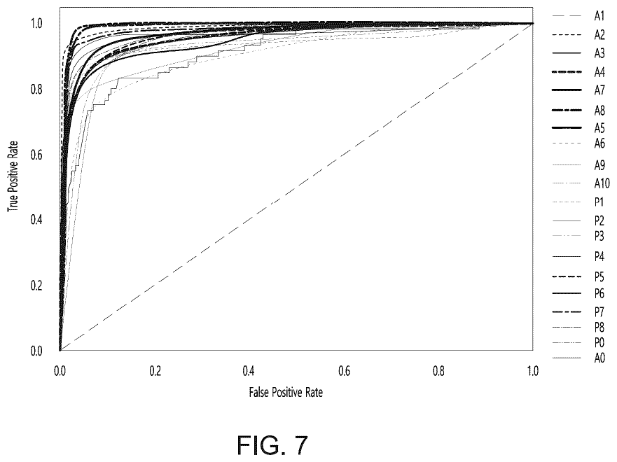

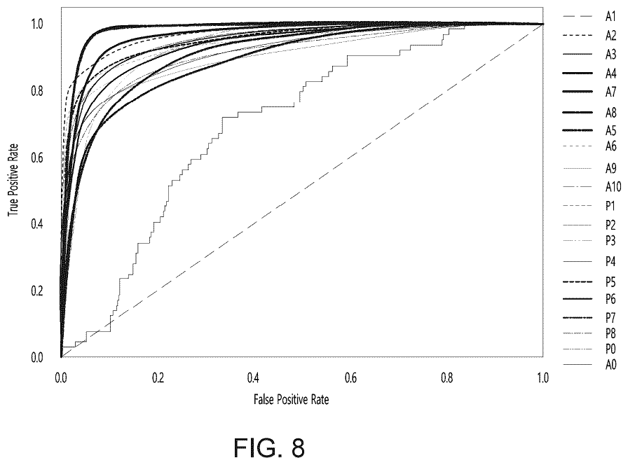

FIGS. 7 and 8 illustrate results of validating performance of identifying stroke patients. FIGS. 7 and 8 show performance of classifying stroke patients on the basis of chunks. FIG. 7 is an ROC curve showing performance for the stroke group. For the stroke group, the micro-average AUC was 0.97; and the macro-average AUC was 0.96. FIG. 8 illustrates an ROC curve showing performance for the ICAS group. For the ICAS group, the micro-average AUC was 0.95, and the macro-average AUC was 0.92. The model for identifying stroke patients showed significantly high classification accuracy overall.

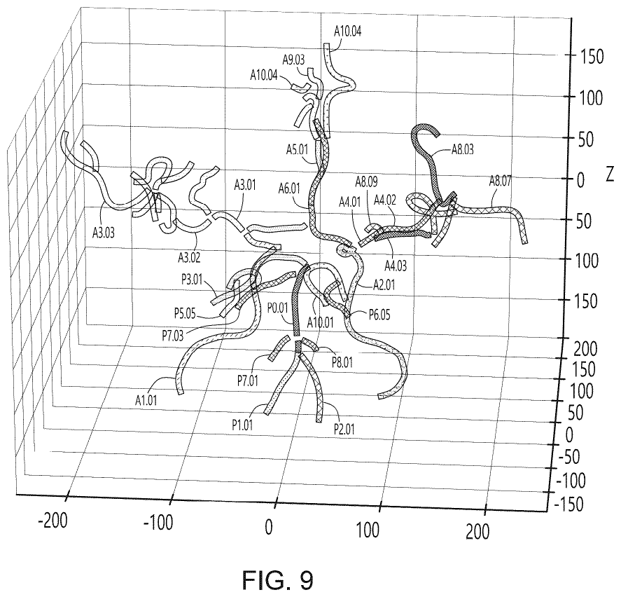

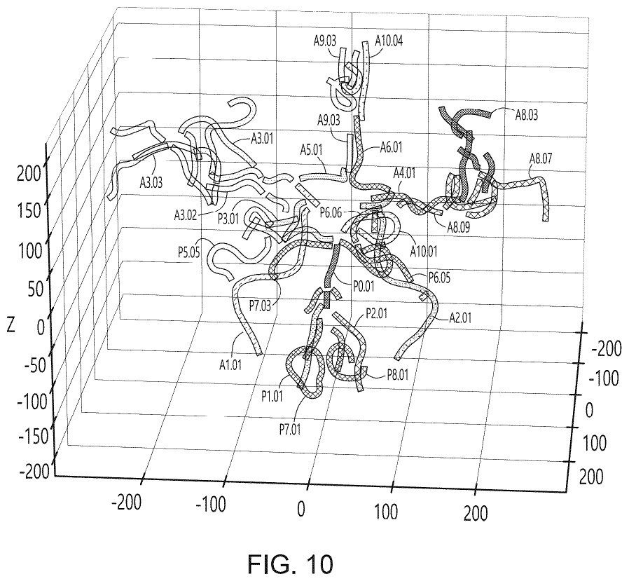

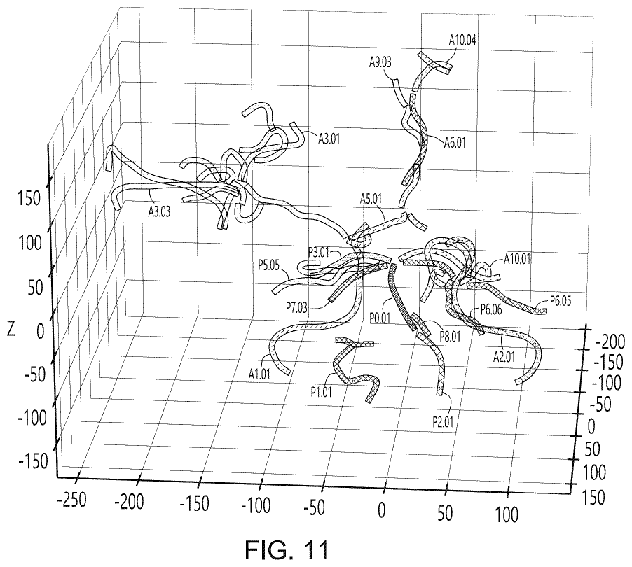

Features of a cerebral artery structure based on cerebral artery branches may be peculiar information of a specific subject. FIGS. 9 to 11 visualize results of classifying cerebral artery branches of the control group, the ICAS group, and the stoke group. 29 cerebral artery branches were extracted from subjects belonging to each group. FIG. 9 shows results of classifying cerebral artery branches of the normal control group. FIG. 10 shows results of classifying cerebral artery branches of the ICAS group. FIG. 11 shows results of classifying cerebral artery branches of the stroke group. FIGS. 9 to 11 show results of visually separating cerebral artery branches in the same coordinate space, and it may be seen that there are regions where the position or shape of a cerebral artery branch(es) differs in the three groups.

Further, a cerebrovascular shape varies widely among individuals. In other words, even when subjects belong to the same group (a normal group, a patient group, or the like), it may not be easy to compare the position of a specific blood vessel in one subject with the position of the corresponding blood vessel in another subject. Accordingly, the researcher proposes a process of uniformly quantifying the above-described features of a cerebral artery structure.

A process in which the analysis device quantifies features of a subject's cerebral artery structure will be described below. Quantization is a process of defining a cerebral artery structure in a standardized space. Further, the analysis device may average the quantified cerebral artery structures of specific subjects belonging to a population to generate a cerebrovascular standard template for the population.

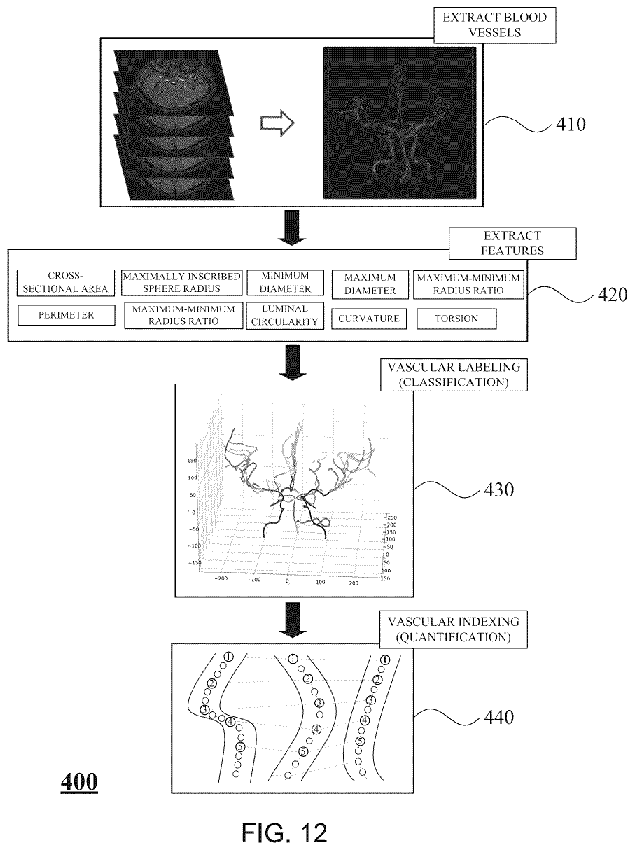

FIG. 12 shows an example of a process 400 of quantifying a cerebrovascular structure. FIG. 12 corresponds to an example of quantifying the cerebrovascular structure of one subject.

The analysis device receives a TOF MRA image of a subject and extracts a vascular structure (410). A vascular structure extraction process is the same as described above.

The analysis device may extract features of the extracted vascular structure (420). Vascular features include a cerebrovascular cross-sectional area, a maximally inscribed sphere radius, a minimum diameter, a maximum diameter, a maximum-minimum radius ratio, a surface circumference (perimeter), a curvature, and luminal circularity.

As described in FIG. 2, the analysis device may label (classify) blood vessels in units of cerebral artery branches on the basis of vascular features (430).

Subsequently, the analysis device indexes corresponding blood vessels in units of the classified vascular structures (e.g., cerebral artery branches) (440). This process corresponds to a vascular structure quantification process. The analysis device may divide a specific classified cerebral artery branch into certain sections and assign indices to the segments or may assign indices to sections at certain intervals. Meanwhile, the structure and length of a specific cerebral artery branch may vary depending on individuals. FIG. 12 illustrates (three) vascular structures having different shapes and lengths for the same cerebral artery branch. This is intended to illustrate that even the same vascular structure unit may have different shapes and lengths. FIG. 12 shows an example of indexing identical cerebral artery branches with 1 to 5. The structure of quantified cerebral artery branches may be evaluated on the basis of the positions of indices.

A process of indexing a specific cerebral artery branch will be described below. The analysis device may classify the specific cerebral artery branch in cerebral vessels of a subject and index the classified specific cerebral artery branch (e.g., A4.01 in Table 1).

A specific vessel branch may be indexed in various ways. The analysis device may divide the specific cerebral artery branch into a certain number of (N) segments. The segments have the same length. The analysis device may assign indices on the basis of a specific point (e.g., the center) in the divided segments. The point where an index is assigned may vary. For example, the point where an index is assigned may be any one of the center, the start, and the end of a segment. It is assumed below that the point where an index is assigned is the center of a segment. The analysis device may assign indices to the N segments on a one-to-one basis. Alternatively, the analysis device may assign indices to the N segments at certain intervals.

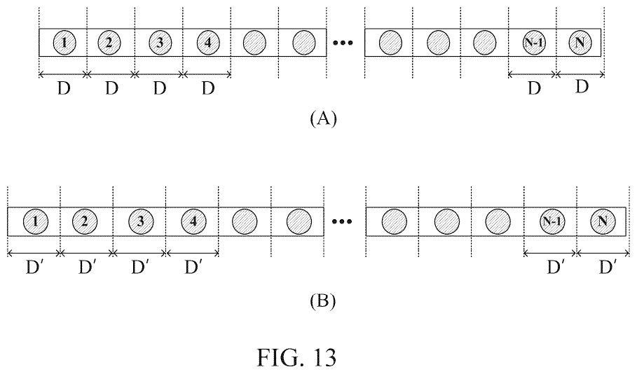

FIG. 13 shows an example of indexing of one vessel branch. FIG. 13 shows an example of a vessel branch with N sections. FIG. 13 shows an example of assigning indices to divided sections. In FIG. 13, a straight blood vessel is assumed for convenience of description. In this case, each segment of the cerebral artery branch may be a region including one spot or a certain number of spots.

FIG. 13 shows examples of assigning indices to identical cerebral artery branches (e.g., A4.01).

FIG. 13A shows an example of assigning indices to a cerebral artery branch of subject A. The analysis device extracts cerebral vessels of subject A and performs classification in units of cerebral artery branches. Subsequently, the analysis device may index the cerebral artery branches. The analysis device may divide the cerebral artery branch (A4.01) of subject A into N segments and assign an index to each segment. FIG. 13A shows an example of dividing the cerebral artery branch of subject A into N segments and then assigning indices. Here, each segment has a length of D.

FIG. 13B shows an example of assigning indices to a cerebral artery branch of subject B. The cerebral artery branch (A4.01) of subject B is longer than the cerebral artery branch of subject A. The analysis device may divide the cerebral artery branch (A4.01) of subject B into N segments and assign an index to each segment. FIG. 13B shows an example of dividing the cerebral artery branch of subject B into N segments and then assigning indices. Here, each segment has a length of D′.

Further, the analysis device may quantify (index) all cerebrovascular structures of a specific population and prepare a cerebrovascular standard template for the population. A cerebrovascular standard template may be provided per vascular region (chunk or cerebral artery branch).

For example, the analysis device may provide a cerebrovascular standard template for a specific group (e.g., stroke group). Here, the stroke group is composed of multiple subjects, and the subjects may have slightly different cerebrovascular shapes. The analysis device may uniformly standardize cerebral artery branches of the stroke group.

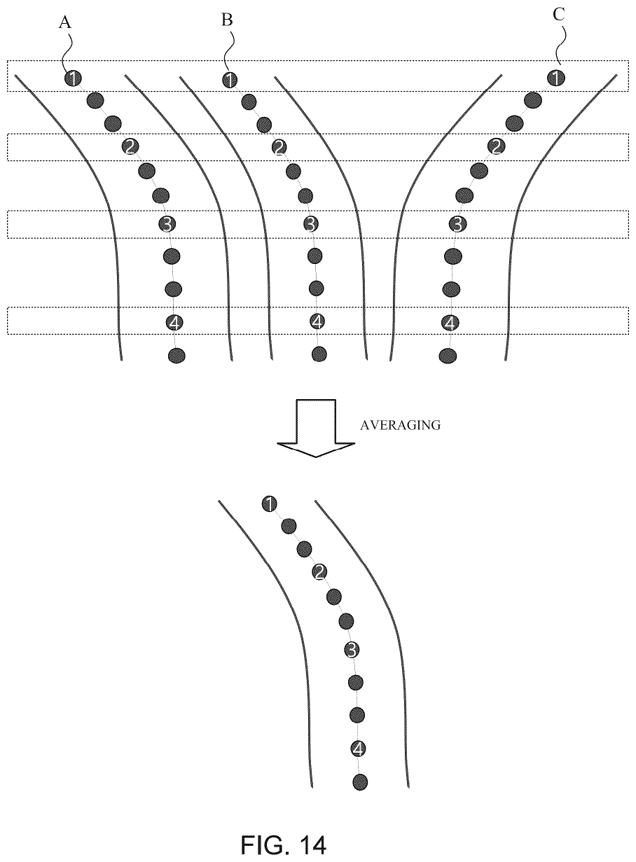

FIG. 14 shows an example of a process of standardizing information on cerebral artery branches. FIG. 14 shows an example of generating a cerebrovascular standard template for identical specific cerebral artery branches. In other words, a plurality of cerebrovascular branches shown in FIG. 14 have the same branch code. In FIG. 14, each circle in blood vessels may be one spot.

The analysis device normalizes identical cerebral artery branches of subjects A, B, and C belonging to the same population. FIG. 14 shows an example of assigning indices at certain segmental intervals. Subjects A, B, and C have slightly different cerebral artery branches. The analysis device may average the positions of identical indices in normalized cerebral artery branches of subjects A, B, and C. For example, the analysis device may adjust the cerebral artery branches of subjects A, B, and C in the same direction, place the adjusted cerebral artery branches in the same coordinate space, and then extract the index position of each branch. The analysis device may average the positions of the identical indices in the cerebral artery branches of subjects A, B, and C. The analysis device may perform the same task for all the cerebral artery branches. This process allows the analysis device to generate a cerebrovascular standard template for a specific population (e.g., healthy males or males with a cerebrovascular disease).

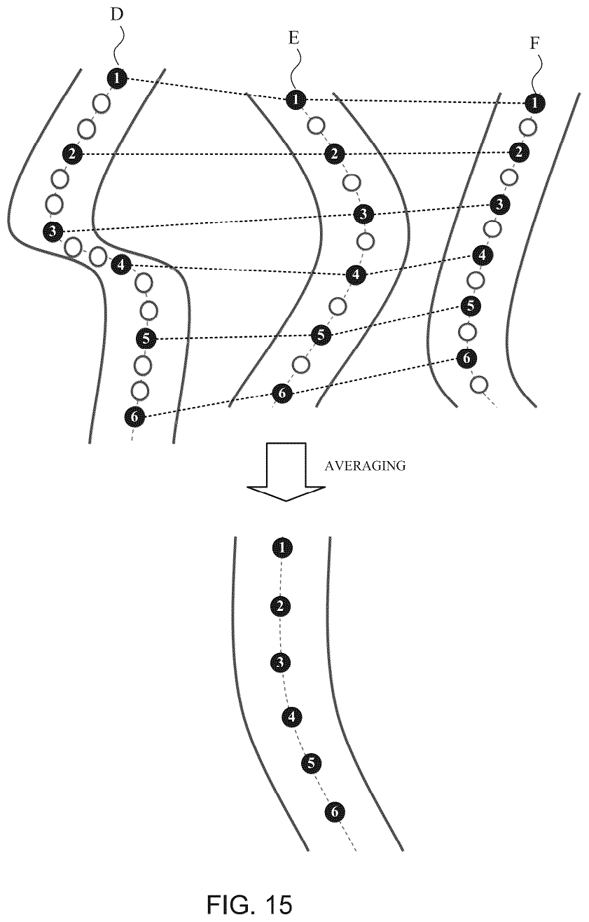

FIG. 15 is a diagram illustrating another example of a process of standardizing information on cerebral artery branches. FIG. 15 shows an example of generating a cerebrovascular standard template for identical specific cerebral artery branches. In FIG. 15, each circle in blood vessels may be one spot.

The analysis device normalizes identical cerebral artery branches of subjects D, E, and F belonging to the same population. FIG. 15 shows an example of assigning indices at certain segmental intervals. Subjects D, E, and F have slightly different cerebral artery branches. The cerebral artery branch of subject D is longer than those of the other subjects. The analysis device may average the positions of identical indices in normalized cerebral artery branches of subjects D, E, and F. For example, the analysis device may adjust the cerebral artery branches of subjects D, E, and F in the same direction, place the adjusted cerebral artery branches in the same coordinate space, and then extract the index position of each branch. The analysis device may average the positions of the identical indices in the cerebral artery branches of subjects D, E, and F. The analysis device may perform the same task for all the cerebral artery branches. This process allows the analysis device to generate a cerebrovascular standard template for a specific population (e.g., healthy males or males with a cerebrovascular disease).

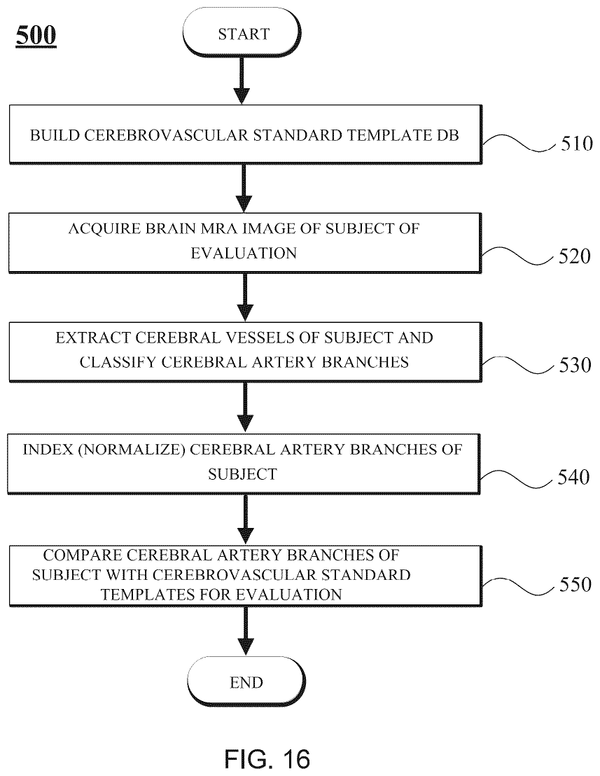

FIG. 16 is a flowchart illustrating a process 500 of evaluating a cerebrovascular structure of a subject using a cerebrovascular standard template.

The analysis device builds a cerebrovascular standard template DB for a specific population (510). A process of building a cerebrovascular standard template is the same as described above in FIGS. 12 to 15. The analysis device may classify and normalize cerebral artery branches of subjects belonging to the population by type and then average index positions to generate a cerebrovascular standard template for the population. Here, the specific population may be defined by sex and health status (presence or absence of a certain brain disease). The cerebrovascular standard template DB is assumed to store a cerebrovascular standard template for a healthy male group, a cerebrovascular standard template for a healthy female group, a cerebrovascular standard template for a male group with the specific brain disease, and a cerebrovascular standard template for a female group with the specific brain disease.

The analysis device acquires a brain MRA image of a subject of evaluation (520). The analysis device may extract cerebral vessels from the brain MRA image of the subject of evaluation and classify cerebral artery branches on the basis of features of the extracted cerebral vessels (530).

The analysis device indexes (normalizes) at least one cerebral artery branch to be analyzed among the cerebral artery branches (540). Here, the analysis device may index all the cerebral artery branches of the subject. Further, the analysis device may selectively index a specific cerebral artery branch(es) which is meaningful to the subject. The latter may be a case where the correlation between a specific disease and a specific cerebral artery branch(es) is identified in advance.

The analysis device may compare all the cerebral artery branches or the specific cerebral artery branch(es) of the subject with a cerebrovascular standard template of the population (550). The analysis device may compare each of the cerebral artery branches of the subject with a cerebrovascular standard template of the corresponding cerebral artery branch. Alternatively, the analysis device may compare the specific cerebral artery branch(es) with a cerebrovascular standard template of the corresponding cerebral artery branch.

For example, when the subject is a male, the analysis device may compare cerebral artery branches of the subject with cerebrovascular standard templates of a normal male group. The analysis device may compare the positions of indices for the cerebral artery branches of the subject with the positions of the same indices for the cerebrovascular standard templates to determine differences between the structures of the cerebral artery branches of the subject and the structures of the cerebrovascular standard templates. When a cerebral artery branch(es) has a difference of a preset threshold or more, the analysis device may output the cerebral artery branch and the difference value. Alternatively, when a cerebral artery branch(es) has a difference of the preset threshold or more, the analysis device may determine that the subject does not belong to a normal category (is highly likely to have a specific disease).

Alternatively, when the subject is a male, the analysis device may compare cerebral artery branches of the subject with cerebrovascular standard templates of a male group with a specific brain disease. The analysis device may compare the positions of indices for the cerebral artery branches of the subject with the positions of the same indices for the cerebrovascular standard templates to determine similarity between the structures of the cerebral artery branches of the subject and the structures of the cerebrovascular standard templates. When a cerebral artery branch(es) has a difference that is smaller than a preset threshold (i.e., has a similar structure), the analysis device may output the cerebral artery branch and the difference value. Alternatively, when a cerebral artery branch(es) has a difference that is smaller than the preset threshold, the analysis device may determine that the subject does not belong to a normal category (is highly likely to have the specific disease).

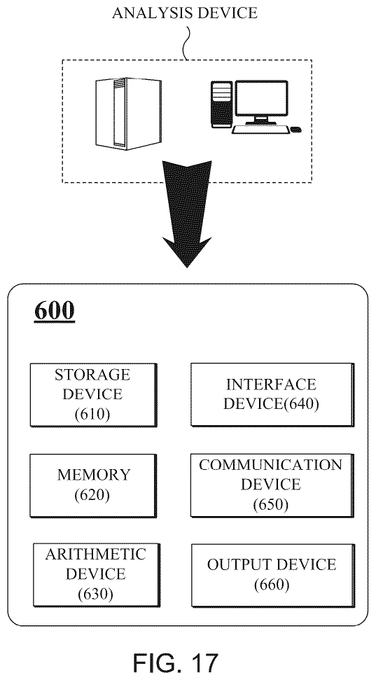

FIG. 17 shows an example of an analysis device 600 for analyzing cerebral artery branches of a subject. The analysis device 600 indexes cerebral artery branches of a subject. The analysis device 600 may evaluate the subject on the basis of the cerebral artery branches and the indices. For example, the analysis device 600 may classify the cerebral artery branches of the subject and evaluate a phenotype of the subject on the basis of the classified cerebral artery branches. The analysis device 600 may classify the cerebral artery branches of the subject and evaluate medication effects on the basis of the classified cerebral artery branches. The analysis device 600 may standardize features of cerebrovascular features on the basis of cerebral artery branches of a specific population.

The analysis device 600 may be physically implemented in various forms. For example, the analysis device 600 may have the form of a computing device such as a PC, a server on a network, a chipset dedicated to data processing, and the like.

The analysis device 600 may include a storage device 610, a memory 620, an arithmetic device 630, an interface device 640, a communication device 650, and an output device 660.

The storage device 610 may store MRA images generated by MRA equipment.

The storage device 610 may store codes or programs for extracting the foregoing cerebrovascular structure (spots, segments, etc.) from MRA images.

The storage device 610 may store codes or programs for extracting feature values (feature vectors) from spots of a cerebrovascular structure. (i) The feature values may include at least one of factors including vascular centerline coordinates, a cerebrovascular cross-sectional area, a maximally inscribed sphere radius, a minimum diameter, a maximum diameter, a maximum-minimum radius ratio, a hydraulic luminal diameter, a perimeter of a vascular cross-section, a surface circumference, torsion, curvature, luminal circularity, and the like. (ii) Further, the feature values may further include brightness values of the corresponding spot areas.

The storage device 610 may store a first learning model that classifies affiliated chunks in units of spots and a second learning model that classifies each of spots belonging to the same chunk into cerebral artery branches, which are described in FIGS. 2 and 3.

The storage device 610 may store reference information for subject evaluation. The reference information is cerebrovascular standard templates of a specific population. Here, the population may vary such as a group of normal subjects, a group of subjects with a specific phenotype, a group of subjects medicated with medicine, and the like. Further, the population may be additionally divided by information such as sex, and the storage device 610 may have a cerebrovascular standard template for each sub-group (normal males, females with a specific cerebrovascular disease, males medicated with medication for a specific disease, and the like).

The memory 620 may store data, information, and the like generated during a process in which the analysis device 600 classifies cerebral artery branches in an MRA image, indexes the cerebral artery branches, and evaluates the indexed cerebral artery branches.

The interface device 640 is a device that externally receives certain instructions and data.

The interface device 640 may receive the MRA image from a physically connected input device or external storage device.

The interface device 640 may receive the cerebrovascular standard templates of the population from the physically connected input device or external storage device.

The interface device 640 may receive an instruction for selecting a specific cerebral artery branch to be analyzed from among the cerebral artery branches.

The interface device 640 may transmit a result obtained by classifying the MRA image of the subject to an external object.

The communication device 650 is a component that receives and transmits certain information via a wired or wireless network.

The communication device 650 may receive the MRA image from the external object.

The communication device 650 may receive the cerebrovascular standard templates of the population from the external object.

The communication device 650 may receive the instruction for selecting the specific cerebral artery branch to be analyzed from among the cerebral artery branches.

The communication device 650 may transmit the result obtained by classifying the MRA image of the subject to an external object such as a user terminal.

The interface device 640 may be a device that transmits data received from the communication device 650 to the inside of the analysis device 600.

The output device 660 is a device that outputs certain information. The output device 660 may output an interface necessary for a data processing process, MRA images, cerebrovascular structures extracted from MRA images, chunk classification, cerebral artery branch classification, cerebral artery branch indices, analysis results based on indexed cerebral artery branches, and the like.

The arithmetic device 630 may classify the cerebral artery branches in the MRA image.

The arithmetic device 630 may reconstruct the structures in the MRA image through geometry processing. Here, the arithmetic device 630 may identify spots, segments, and the like which are the foregoing cerebrovascular structures.

The arithmetic device 630 may divide a continuous 3D space in the MRA image into a plurality of cells on the basis of vertices of isosurfaces. In this process, the arithmetic device 630 may perform a preprocessing process such as noise removal, normalization, and the like of the image. The arithmetic device 630 may divide a structure into a plurality of cells constituting the vascular surface, and extract the main arterial centerline from the perimeter surface of each cell in the cerebrovascular MRA image.

The arithmetic device 630 may divide the vascular surface into cells having a certain size in the cerebrovascular MRA image and extract the starting point and skeleton of the centerline of a brain artery on the basis of the vascular surface. The arithmetic device 630 may perform vascular skeleton refinement to make the end point of the centerline more distinct. The arithmetic device 630 may (i) skeletonize the cerebrovascular regions and surfaces, (ii) prune branches under a predetermined threshold, (iii) generate a linked list of tree structures on the basis of the refined skeletal structure, and (iv) determine the end point by designating a leaf node from the linked list. The analysis device may extract the centerline of the blood vessel by tracing cell interfaces connecting the determined start point and end point.

The arithmetic device 630 may identify spots, which are basic units of cubic cells of a 3D cerebral artery tree having certain intervals from the centerline of an artery. In addition, the arithmetic device 630 may identify a segment that is a specific region in which multiple spots are divided on the basis of a bifurcation in a vascular structure.

The arithmetic device 630 may input the spots extracted from the MRA image into the first learning model one by one to classify the input spots into chunks. The arithmetic device 630 may extract feature values of the spots and input the extracted feature values to the first learning model. The operation and learning process of the first learning model are the same as described above. The first learning model may be a DNN-based model. Through this process, an affiliated chunk is determined for each individual spot.

Also, the arithmetic device 630 may determine a final classification result in units of segments. As described above, the arithmetic device 630 may set a value of the most classified results on the basis of a result obtained by classifying spots belonging to the same segment into a chunk, as a result of chunk classification of spots belonging to the corresponding segment.

The arithmetic device 630 may input spots belonging to the same chunk into the second learning model one by one to classify the spots into cerebral artery branches. The arithmetic device 630 may extract feature values of the spots and input the extracted feature values into the second learning model. The operation and learning process of the second learning model are the same as described above. The second learning model may be a DNN-based model. The second learning model may be an ensemble model employing different types of learning models.

In addition, the arithmetic device 630 may determine a final classification result in units of chunks. As described above, the arithmetic device 630 may set a value of the most classified results on the basis of cerebral artery branch classification results of spots belonging to the same chunk, as a result of cerebral artery branch classification of the spots belonging to the corresponding chunk.

Further, the arithmetic device 630 may validate or correct the cerebral artery branch classification results (primary classification) for the spots in a different method. For example, the analysis device may validate whether segments (branches belonging to the corresponding segments) of which branches are clearly distinguished as left and right or up and down in the image data are in opposite directions on the basis of their 3D coordinates, and correct the classification information when the segments are opposite to each other.

Through this process, the arithmetic device 630 may classify the structures in units of cerebral artery branches extracted from the MRA image.

Meanwhile, the arithmetic device 630 may identify whether the subject is a patient with a specific brain disease on the basis of the MRA image. In this case, the arithmetic device 630 uses a model pretrained for brain disease classification. This has been mentioned in the above-described external validation process. The arithmetic device 630 may extract spots from the MRA image and input feature values of the spots into a brain disease classification model to identify whether the subject of the corresponding image is a patient with the specific brain disease.

A learning model for classifying brain diseases may be a two-stage model. In other words, a first learning model may classify chunks of spots extracted from an MRA image, and a second learning model may receive feature values of the spots in units of chunks and finally determine whether a corresponding subject has a brain disease. In this case, the second learning model may receive feature values in units of spots and determine whether the corresponding subject has a brain disease on the basis of evaluation results of all the spots. Alternatively, the second learning model may receive feature values of all spots of the same chunk at once to determine whether the corresponding subject has a brain disease. The second learning model may be a model that extracts vessel branch characteristics of spots or each spot and determines whether the corresponding subject has a brain disease on the basis of the extracted characteristics. This is determined in accordance with a process of training the second learning model and the training data.

The arithmetic device 630 may normalize at least some of the classified cerebral artery branches. The arithmetic device 630 may normalize all the cerebral artery branches of the subject or only a specific cerebral artery branch(es) to be analyzed.

Further, the arithmetic device 630 may index cerebral artery branches of subjects belonging to a specific population and generate cerebrovascular standard templates of the population. A cerebrovascular standard template generation process is the same as described above in FIGS. 12 to 15.

The arithmetic device 630 may compare the indexed cerebral artery branch(es) of the subject with the cerebrovascular standard template to evaluate the subject's cerebrovascular structure. This process is the same as described above in FIG. 16.

The arithmetic device 630 may be a device such as a processor that processes data and certain operations, an application processor (AP), or a chip in which a program is embedded.

The above-described image processing method, MRA-based cerebral artery analysis method, cerebral artery branch classification method, cerebrovascular standard template generation method, and brain disease classification method may be implemented as a program (or application) including an algorithm executable on a computer. The program may be stored and provided in a transitory or non-transitory computer-readable medium.

A non-transitory computer-readable medium is not a medium that stores data for a short moment, such as a register, a cache, or a memory, but a medium that stores data semi-permanently and is readable by a device. Specifically, the various foregoing applications or programs may be stored and provided in a non-transitory computer-readable medium, such as a compact disc (CD), a digital video disc (DVD), a hard disk, a Blu-ray disc, a Universal Serial Bus (USB) memory, a memory card, a read-only memory (ROM), a programmable read-only memory (PROM), an erasable PROM (EPROM), an electrically EPROM (EEPROM), a flash memory, or the like.

The transitory computer-readable medium is any of various random access memories (RAMs) such as a static RAM (SRAM), a dynamic RAM (DRAM), a synchronous DRAM (SDRAM), a double data rate SDRAM (DDR SDRAM), an enhanced SDRAM (ESDRAM), a Synclink DRAM (SLDRAM), and a direct Rambus RAM (DRRAM).

The above-described technology can be used for quantifying, evaluating, and predicting cerebrovascular diseases by classifying cerebrovascular structures in units of cerebral arterial regions. According to the above-described technology, it is possible to evaluate cerebrovascular structures which vary depending on individuals, by quantifying cerebrovascular branches. According to the above-described technology, it is possible to standardize cerebrovascular structures of a specific population.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of generating standardized cerebrovascular structure information, the method comprising:
receiving, by an analysis device, a cerebrovascular image of a subject;
extracting, by the analysis device, a plurality of vascular unit structures from the cerebrovascular image;
extracting, by the analysis device, feature values of each of the plurality of vascular unit structures;
classifying, by the analysis device, each of the plurality of vascular unit structures into a chunk by inputting the feature values of each of the plurality of vascular unit structures into a pretrained first learning model;
classifying, by the analysis device, multiple vessel branches composed of vascular unit structures belonging to the same chunk by inputting feature values of each of the vascular unit structures belonging to the same chunk into a pretrained second learning model; and
dividing, by the analysis device, at least one of the multiple vessel branches into a predetermined number of segments and setting indices for all the segments or segments at certain intervals among the segments.

2. The method of claim 1, wherein the vascular unit structures are spots, and
the spots are cubic cells having certain intervals in an arterial centerline extracted from the cerebrovascular image.

3. The method of claim 1, wherein the feature values include a cerebrovascular cross-sectional area, a maximally inscribed sphere radius, a minimum diameter, a maximum diameter, a maximum-minimum radius ratio, a surface circumference, torsion, curvature, and luminal circularity.

4. The method of claim 1, wherein the classifying of each of the plurality of vascular unit structures into the chunk comprises:
performing, by the analysis device, primary chunk classification of each of the plurality of vascular unit structures using the first learning model; and
performing, by the analysis device, secondary chunk classification of vascular unit structures belonging to the same segment in a majority voting manner on the basis of results of the primary chunk classification of the vascular unit structures belonging to the same segment among the plurality of vascular unit structures, and
the segment is composed of vascular unit structures belonging to a region divided by a bifurcation in a vascular structure.

5. The method of claim 1, wherein the classifying of the multiple vessel branches comprises:
performing, by the analysis device, primary vessel branch classification of each of the plurality of vascular unit structures belonging to the same chunk using the second learning model; and
performing, by the analysis device, secondary vessel branch classification of the vascular unit structures belonging to the same chunk in a majority voting manner on the basis of results of the primary vessel branch classification of the vascular unit structures belonging to the same chunk among the plurality of vascular unit structures, and
the segments are composed of vascular unit structures belonging to a region divided by a bifurcation in a vascular structure.

6. A method of generating standardized cerebrovascular structure information, the method comprising:
receiving, by an analysis device, cerebrovascular images of subjects belonging to a population;
setting, by the analysis device, indices for at least one vessel branch of each of the subjects using the cerebrovascular images of the subjects; and
generating, by the analysis device, cerebrovascular structure information of the population by averaging positions of identical indices in the at least one vessel branch of each of the subjects,
wherein the setting of the indices for the at least one vessel branch of each of the subjects comprises:
classifying, by the analysis device, each of a plurality of vascular unit structures extracted from the cerebrovascular images into a chunk by inputting feature values of each of the plurality of vascular unit structures into a pretrained first learning model;
classifying, by the analysis device, the at least one vessel branch composed of vascular unit structures belonging to the same chunk by inputting feature values of each of the vascular unit structures belonging to the same chunk into a pretrained second learning model; and
dividing, by the analysis device, the at least one vessel branch into a predetermined number of segments and setting indices for all the segments or segments at certain intervals among the segments.

7. An analysis device for evaluating a subject using standardized cerebrovascular structure information, the analysis device comprising:
an input device configured to receive a cerebrovascular image of a subject;
a storage device configured to store a first learning model which classifies vascular unit structures into chunks, a second learning model which classifies cerebrovascular branches having vascular unit structures belonging to the same chunk, and standardized cerebrovascular structure information of a population; and
an arithmetic device configured to extract a plurality of vascular unit structures from the cerebrovascular image on the basis of geometric features of a three-dimensional (3D) model, classify the plurality of vascular unit structures into chunks by inputting feature values of each of the plurality of vascular unit structures into the first learning model, classify multiple vessel branches composed of the vascular unit structures belonging to the same chunk by inputting feature values of each of the vascular unit structures belonging to the same chunk to the second learning model, assign indices for dividing vascular units belonging to at least one of the multiple vessel branches at identical intervals, and compare a position of an index for the at least one vessel branch of the subject with an index position of the standardized cerebrovascular structure information.

8. The analysis device of claim 7, wherein the vascular unit structures are spots, and
the spots are cubic cells having certain intervals in an arterial centerline extracted from the cerebrovascular image.

9. The analysis device of claim 7, wherein the feature values include a cerebrovascular cross-sectional area, a maximally inscribed sphere radius, a minimum diameter, a maximum diameter, a maximum-minimum radius ratio, a surface circumference, torsion, curvature, and luminal circularity.

10. The analysis device of claim 7, wherein the arithmetic device performs primary chunk classification of each of the plurality of vascular unit structures using the first learning model and performs secondary chunk classification of vascular unit structures belonging to the same segment in a majority voting manner on the basis of results of the primary chunk classification of the vascular unit structures belonging to the same segment among the plurality of vascular unit structures, and
the segment is composed of vascular unit structures belonging to a region divided by a bifurcation in a vascular structure.
